(19) United States
(12) Patent Application Publication
(10) Pub. No.: US 2017/0340336 A1
(43) Pub. Date: Nov. 30, 2017

(54) CLOSURE DEVICE SUITABLE FOR CLOSING THE ATRIAL APPENDAGE

(71) Applicant: Peter Osypka Stiftung, Grenzach-Wyhlen (DE)

(72) Inventor: Peter Osypka, Grenzach-Wyhlen (DE)

(21) Appl. No.: 15/532,792

(22) PCT Filed: Nov. 2, 2015
(86) PCT No.: PCT/EP2015/075425

§ 371 Date: Jun. 2, 2017

(30) Foreign Application Priority Data
Dec. 3, 2014 (EP) 14004067.6

(51) Int. Cl. A61B 17/12; B33Y 80/00; B33Y 10/00; A61B 17/00; G06F 19/00; B33Y 70/00; B33Y 50/00; B22F 3/105

(52) U.S. Cl. CPC A61B 17/12172; A61B 17/12136; B33Y 10/00; B33Y 80/00; A61B 17/0057; A61B 17/12122; A61B 2017/00623; A61B 2017/00592; A61B 2017/00588; A61B 2017/00526; B33Y 70/00; B33Y 50/00; A61B 2017/00867; B22F 3/1055; B22F 2003/1057; G06F 19/321; G06F 19/3481; G06F 19/3437

(57) ABSTRACT

A medical closure device that is configured to be inserted into a patient's LAA (left atrial appendage) cavity comprises a closure member and at least one fixing member, wherein the closure member comprises a lid part and a body part one following the other in a proximal-distal direction. The lid part comprises a cover portion and a ring portion, the ring portion being configured to be circumferentially disposed along the rim of an individual LAA ostium, thus sealing the LAA ostium completely. The medical closure device is designed individually for each patient. A method of manufacturing the medical closure device includes the step of forming an LAA model based on computer graphic data of the patient. The LAA model is made by 3-D printing and is an exact reproduction of the patient's LAA. The medical closure device is then preformed using the LAA model.

### BACKGROUND OF THE INVENTION

This invention relates to an individually designed medical closure device suitable to be deliverable transluminally via a catheter for placement within and against the left atrial appendage (LAA), and to a method of manufacturing said medical closure device

Left atrial appendage closure (LAAC) is a known treatment strategy for reducing the risk of left atrial appendage blood clots entering the bloodstream and causing a stroke in patients with atrial fibrillation (AF). In atrial fibrillation, over 90% of stroke-causing clots are formed in the left atrial appendage (LAA).

The risk factor for strokes in case of atrial fibrillation also seems to be dependent on the LAA morphology. The classification of LAA morphology includes different types. The major left atrial appendage types are: the “Windsock Type”, the “Chicken Wing Type” and the “Broccoli Type”. The orifice of the LAA to the left atrium is called the LAA ostium. The LAA ostium is also irregularly shaped, having a circular, elliptical or elongated shape with a more or less wavy margin.

Due to the extremely complex and heterogeneous morphology of the LAA and the irregular wavy shape of the LAA ostium, the implantation of an LAA closure device is a great challenge requiring an enormous skill.

The European patent EP 2 074 953 B1 describes a vascular occlusion device for occlusion of an atrial appendage. The medical device includes a first portion having at least one plane of occlusion that is configured to be positioned outside of the left atrial appendage, and a second portion having at least one plane of occlusion that is configured to be at least partially positioned within a cavity defined by the left atrial appendage. The second portion comprises a cylindrical surface formed of a metallic fabric comprising a plurality of wire strands configured to engage the cavity wall along the entire length of the second portion. A plurality of hooks extend from the second portion. The hooks engage the cavity within the LAA.

Currently two kinds of LAA occluder are clinically used, the Watchman system and the ACP (Amplatzer Cardiac Plug) System.

### SUMMARY OF THE INVENTION

The problem to be solved by the present invention is to provide a left atrial appendage closure device which is individually designed for each and every patient. The closure device should be sized according to the patient's LAA. The closure device should thus be optimally adapted to the morphology of the LAA and of the LAA ostium so that the closure device fits exactly and properly in order to seal the LAA ostium completely. Furthermore, the closure device should offer safe and easy insertion. After insertion the closure device should be fixable easily and reliably.

This problem is solved by an individually designed closure device comprising a closure member and at least one fixing member. The closure member comprises a lid part and a body part one following the other in a proximal-distal direction. The lid part is configured to cover and seal the LAA ostium. The lid part thus comprises a cover portion and a ring portion. The ring portion is configured to be circumferentially disposed along the rim of each individual LAA ostium thus sealing the LAA ostium. The ring portion of the lid is identical in shape to the rim of the LAA ostium thus forming a lid that fits correctly. The body of the closure member is configured to be positioned within the LAA cavity.

The lid part of the closure member is an important aspect of the closure device. The lid part is formed according to the patient's LAA ostium and must come into direct and close contact with the tissue of the LAA ostium after the device is inserted, deployed and positioned in the LAA cavity. The close contact to the tissue of the LAA ostium is compulsory in order to attain an excellent seal. The body of the closure member is positioned inside the LAA cavity.

In the delivery configuration the closure device is compressed inside a delivery catheter. After the delivery catheter is retracted the closure device expands.

### Material

The closure device may be made of any material suitable to be implanted in a human or animal body and suitable to be deliverable over a catheter for placement within and against an LAA. The closure device may be made of metal or of bio-compatible polymers or a combination thereof.

The closure device is preferably made of a material showing shape memory properties. Thus, the closure member can be formed or pre-formed to the desired shape in a thermal tempering treatment.

The term “shape memory material” is used herein to refer to a material which recovers from a deformed shape to a pre-formed shape. The shape memory material may be a shape memory alloy, a shape memory steel alloy or shape memory polymer. Shape memory materials are known. The most preferred shape memory metal is Nitinol. In one embodiment the closure device is thus formed of Nitinol.

The shape memory material may be covered with a precious metal like gold or platinum.

Representative shape-memory polymers are e.g. polyurethanes, polyethylene terephthalate (PET), polyethylene oxides (PEO) or block copolymers containing a silicone segment. The shape memory polymer may be a carrier for magnetic nanoparticles.

In one embodiment the fixing member is made of a material showing shape memory properties, preferably the same material as the closure member.

In one embodiment the fixing member is made of polymers. The fixing member is e.g. a T-bar.

The tension member may be made of any elastic material suitable to be implanted in a human or animal body. Preferably the tension member is made of the same material as the closure member and/or the fixing member.

In one embodiment the closure device (including the closure member, tension member and fixing member) is made of Nitinol.

Closure member

An important aspect of the invention resides in the fact that the closure device and especially its closure member is sized according to the patient's LAA. By using an LAA model to form the closure member—as outlined below—it was possible for the first time to provide a closure member, whose lid part has exactly the same irregular wavy shape as the LAA ostium and whose body part has the same irregular shape as the inside of the LAA cavity.

As the sealing of the LAA is mainly attained by sealing the ostium, the sealing performance of the body of the closure member can vary. After the closure device has been inserted into the LAA, the body of the closure member adjoins the tissue of each individual LAA cavity wall either with direct contact to the tissue or in a non-contact manner. In one embodiment the body of the closure member is identical in shape and size to the patient's LAA cavity thus being configured to adjoin the tissue of the LAA cavity directly.

The closure member may have any texture or structure. In one embodiment the closure member is a metal braid, preferably a Nitinol braid. In one embodiment the closure member is comprised of a metal grid or metal lattice. In one embodiment the closure member comprises a body part in the form of a polymer balloon and a lid part made of polymer or of a metal braid.

By way of a first example and without limiting the structure of the closure member, it is described herein to be a braid. The invention will be explained in a first example using a braid as a closure member.

The braid is preferably made of Nitinol and comprises a multiplicity of wire elements, preferably at least 30 wire elements, more preferably 30 to 100 wire elements having a diameter of about 0.03 to 0.15 mm. Wire elements may be wires, filaments, threads or the like.

The ends of the wires of the braid are brought together and held by a closure assembly, said closure assembly forming the distal end of the closure member. Any closure assembly capable of preventing the wires from escaping may be used. Non-limiting examples are a sleeve or a ring to which the ends are welded or an assembly in which the ends are welded together to form a welded ball.

The braid may be self-expanding and thus may have a first compressed state and a second expanded state. The braid is preferably made in one piece having a lid part and a body part.

The braid may further comprise at least one layer of occluding material in the form of a coating or a patch. The coating or the patch improves the sealing performance.

The coating may be on the outside and/or on the inside of the braiding. The coating is suitably made of silicone, polyurethane, polyester such as PET or polytetrafluoroethylene (PTFE).

In the delivery configuration the braid is compressed inside the delivery catheter. After the delivery catheter is retracted the braid expands radially due to the elasticity of the braid and recovers to its pre-formed state due to its shape memory property.

After the closure device is manufactured the completed braid may—after being inserted into the LAA cavity—be filled with a plastic, e.g. silicone or polyurethane, in order to improve the stability of the closure member or to provide a drug carrier. The drug carrier allows the release of any drug, e.g. blood thinners.

By way of a second example the closure member is described to be a polymer balloon having a lid. The balloon forming the body of the closure member is preferably made of silicone and can—after being inserted into the LAA cavity—be filled with liquid or foam (silicone foam or polyurethane foam). The filling ensures that the balloon is pressed against the tissue of the LAA cavity. The lid may be a metal braid or a lid made of a polymer, e.g. made of silicone.

In the delivery configuration the balloon with the lid is compressed inside the delivery catheter. After the delivery catheter is retracted the balloon expands.

### Fixing Member

The fixing members are necessary to engage the closure member after insertion of the closure device in order to retain the closure device within the LAA cavity. Due to the extremely complex and heterogeneous morphology of the LAA—as outlined above—it is not at all easy to achieve a safe fixing without penetrating the soft wall of the LAA. Therefore the LAA model also forms the basis for determining the shape of the fixing member.

The fixing member is tailored to the closure member. In a first example a fixing member tailored to a metal braid is described. If the closure member is a braid of metal wires, the fixing member preferably comprises at least one wire element or tape element preferably made of the same material as the braid. The fixing element may be hook shaped at its distal end. Hook shaped means said element bends toward the inner wall of the LAA cavity after the closure device has been inserted. Any kinds of hooks are conceivable. Other kinds of known fixing members such as loops, T-bars or coils of the screw in type may also be used.

Preferably, the fixing element comprises 2 to 10 wire elements, more preferably 3 to 6 wire elements, connected to and preferably grouped evenly around the distal end of the body part of the closure member. If for example four wires are present, the distance between the wires is preferably about 90 degrees.

The length of the fixing members depends on the length and morphology of the LAA cavity. Thus, the LAA model also forms the basis for determining the length of the fixing members. It must be ensured that the LAA inner wall is reached by the fixing members. In one embodiment the closure device comprises a fixing element wherein the wire elements of the fixing element have different lengths with respect to one another.

In a second example the fixing member is tailored to a closure member made of a polymer. In this case the fixing member is preferably a polymer T-bar.

### Tension Member

In order to achieve safe fixing, an elastic element regulating the fixing in terms of its restoration force would be an advantage. In one embodiment the closure member itself is sufficiently elastic and the fixing member is thus directly attached to the distal end of the closure member. In another embodiment a tension member is placed between the closure member and the fixing member.

The tension member ensures improved pressing of the closure member against the LAA and thus ensures a safe closure of the LAA. The tension member also supports keeping the closure device inside the LAA.

A tension member is provided especially if the closure member is a braid.

If a tension member is present, the fixing member is attached to the distal end of the tension member.

The tension member may be a coil spring, preferably made of the same material as the braid. Preferably the coil spring is made of Nitinol and cut out of a tube-like piece of Nitinol. The fixing members may also be cut out of said tube-like piece of Nitinol.

The closure member may have a central lumen extending through the lid part and the body part with a valve placed therein, said valve sealing the lumen. Such a lumen may be advantageous if a guide wire should be inserted so as to facilitate insertion of the closure device into the LAA cavity. If a tension member is present, the lumen extends through the closure member and the tension member.

The closure member may further comprise a thread or double thread leading to the outside. The thread is attached to the proximal end of the closure member and leads to the proximal end of the handle of the delivery catheter. The thread is suitable for re-positioning the closure device if necessary. The thread can be easily removed just by pulling.

In a preferred embodiment the whole closure device is made of Nitinol. The closure device comprises a closure member, a fixing member and a tension member, wherein the closure member is a braid comprising at least 30 wire elements having a diameter of about 0.03 to 0.15 mm, said braid having a lid part and a body part. The fixing member comprises 2 to 10 wire elements being grouped evenly around the distal end of the tension member. The tension member is a coil spring, said coil spring being attached to the distal end of the body part of the closure member. The braid is so configured as to conform in size and shape to the patient's LAA. The braid together with the tension member may have a central lumen.

### Method for Manufacturing the Device

The invention also relates to a method for manufacturing the closure device using 3-D computer graphics.

The method comprises the following steps:
a) generating a three-dimensional computer graphic of the patient's left atrial appendage (LAA) based on medical images of the LAA,
b) transferring the data of the computer graphic to a 3-D printer device,
c) forming a LAA model by 3-D printing, said model being an exact reconstruction of the patient's LAA,
d) manufacturing the closure device by pre-fabricating a closure member comprising a lid part and a body part one following the other in a proximal-distal direction, and pre-fabricating a fixing member and forming at least the lid part of the closure member according to the LAA model obtained in step c) to obtain a pre-formed closure device with at least the lid part of the closure member conforming in size and shape to the patient's LAA ostium; and attaching the fixing member to the closure member either before forming the closure member or thereafter.

The term “medical image” refers to images from various sources such as X-ray, CT scans, MRI scans, ultrasound, etc.

The three dimensional computer graphic is created using common CAD software. Preferably, the medical images are produced in accordance with a display standard such as the DICOM standard (digital imaging and communication in medicine). The DICOM standard image is then exported to the 3-D printer to form an LAA model which corresponds to the patient's LAA. The LAA model may be formed of polymer or of metal. Suitable materials for 3-D printing are known.

In one embodiment the LAA model is made by laser printing and the model is thus made of metal. Any material suitable for laser printing can be used. In one embodiment the LAA model is made of steel. The material must be heat resistant up to the temperature required for shaping the closure device. In another embodiment the LAA model is made by 3-D polymer printing and the LAA model is thus made of polymer.

The forming of the closure device according to the LAA model and thus corresponding to the patient's LAA is an important aspect of the process. The basis of the process is always the medical image and the LAA model gained therefrom. The forming step itself depends on the material used for the closure device.

### Forming a Device of Shape Memory Alloy

If the closure device is made of a shape memory alloy, e.g. made of Nitinol, the process further comprises the following heat treating step:
e) heat-treating the LAA model together with the closure member or closure device placed therein to the transformation temperature of the shape memory alloy to obtain a pre-formed closure device with at least the lid part of the closure member conforming in size and shape to the patient's LAA ostium.

If the shape memory alloy is Nitinol, the heating is up to 400 to 600° C.

The closure device can be pre-formed by pre-forming the closure member and the fixing member separately or the closure member can first be attached to the fixing member or to the tension member having the fixing member at its distal end and then be pre-formed by heat-treating.

The pre-forming of the fixing member has two aspects. In one aspect the term “pre-forming” means adapting the length of the wire elements of the fixing member according to the branches in the patient's LAA cavity. In another aspect “pre-forming” refers to the hook shaping the distal ends of the wire elements of the fixing members.

If both the lid part and the body part of the closure member should be formed according to the patient's LAA, the closure member is advantageously filled with a heat resistant material such as ceramic wool before pressing the closure member into the LAA model. After heat treating the ceramic wool is removed. Due to the filling material the body part of the closure member is pressed against the wall of the LAA model during the heat treatment leading to a pre-formed body part, pre-formed such that the body part comes to bear against the wall of the LAA cavity after the closure device is inserted into the LAA and expanded. The body part of the closure member thus also corresponds to the LAA cavity.

### Modified Step d) Would Read:

d) manufacturing the closure device by pre-fabricating a closure member comprising a lid part and a body part one following the other in a proximal-distal direction, and pre-fabricating a fixing member, filling the closure member with a heat resistant material and forming the closure member by placing it into the LAA model obtained in step c) to obtain a pre-formed closure device which conforms in size and shape to the patient's LAA ostium and the LAA cavity; and attaching the fixing member to the closure member either before forming the closure member or thereafter.

Modified step d) will lead to a closure device with improved fitting.

An important aspect of the shaping process is the fact that medical images of the LAA are used to shape the closure device.

The above-described process of manufacturing an LAA model and pressing the closure device into the model is considered to be the best mode for forming an advantageous process for forming a metal braid. However, there are other conceivable operations leading to a shaping of the closure device by using medical images of the LAA. It is conceivable that a metal ring is shaped according to the image of the LAA ostium and that the proximal area of the closure member is fixed to the metal ring, thus being shaped according to the patient's LAA ostium.

### Forming a Device of Polymer

If the closure device is made of polymer, e.g. of silicone, the body in the form of a balloon and the lid may be formed separately in a known manner using the LAA model as a basis. The lid may be glued onto the balloon. The closure device may also be braided of polymer filaments and then formed.

### Method of Implanting the Device

The method of implanting the closure device in the left atrial appendage may comprise the following steps:
a) providing a delivery catheter having the above-described closure device carried therein,
b) advancing the deliver catheter into the LAA cavity and positioning it therein,
c) releasing the closure device from the delivery catheter, with the closure device expanding to its pre-formed shape,
d) placing the closure device, and
e) removing the delivery catheter.

### BRIEF DESCRIPTION OF THE DRAWINGS

The invention will be explained further by the drawings showing an illustrative but not limitative example of the invention. The reference numbers of the drawings are:

1 Closure member, braided core

2 Fixing member

3 Lid part of the closure member

4 Body part of the closure member

5 Tension member

6 Cover portion of the lid part of the closure member

7 Ring portion of the lid part of the closure member

8 Wire elements

9 Double filament, positioning thread

10 LAA model

11 Lid of the LAA model

12 Rod to secure the lid of the LAA model

13 Guide wire

14 Connection part of the braided wires

15 Semicircular hook

16 Barbed hook

17 Ceramic wool

18 Rim of the LAA model

19 Inner wall of the LAA model cavity

20 Patient's LAA

21 Patient's LAA cavity

22 Patient's LAA ostium

23 Patient's LAA cavity upper part

24 Central lumen

25 Valve

26 Additional closure disc

27 Screw in type coil

28 T-bar

29 Balloon

30 Foam

31 Delivery catheter

32 Pusher

33 Adhesive layer

34 Protective layer

35 Ring

36 Bearing

37 Connector

100 LAA

101 Septum

102 Left atrium

### FIGURES

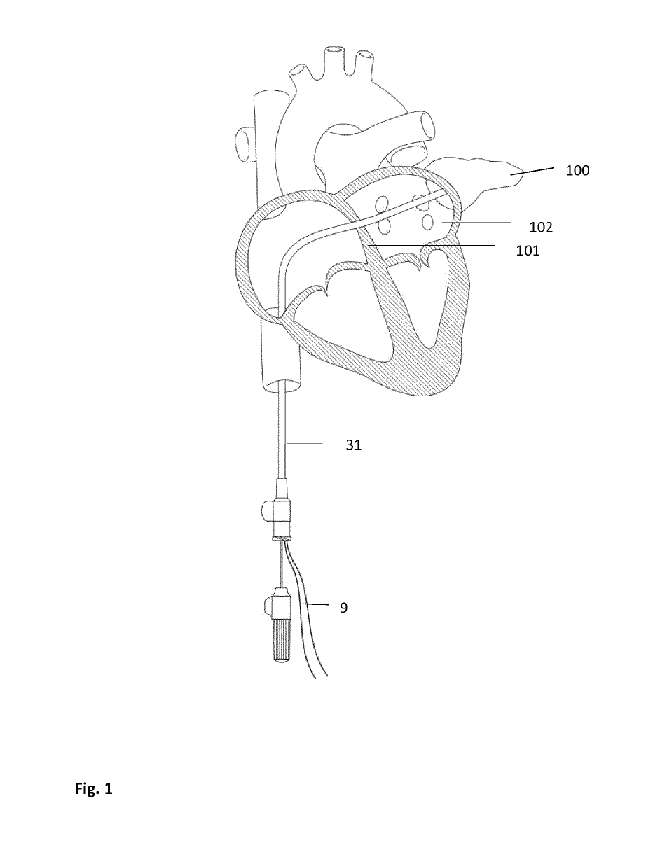

FIG. 1 is an overview drawing showing the transseptal access to a patient's LAA.

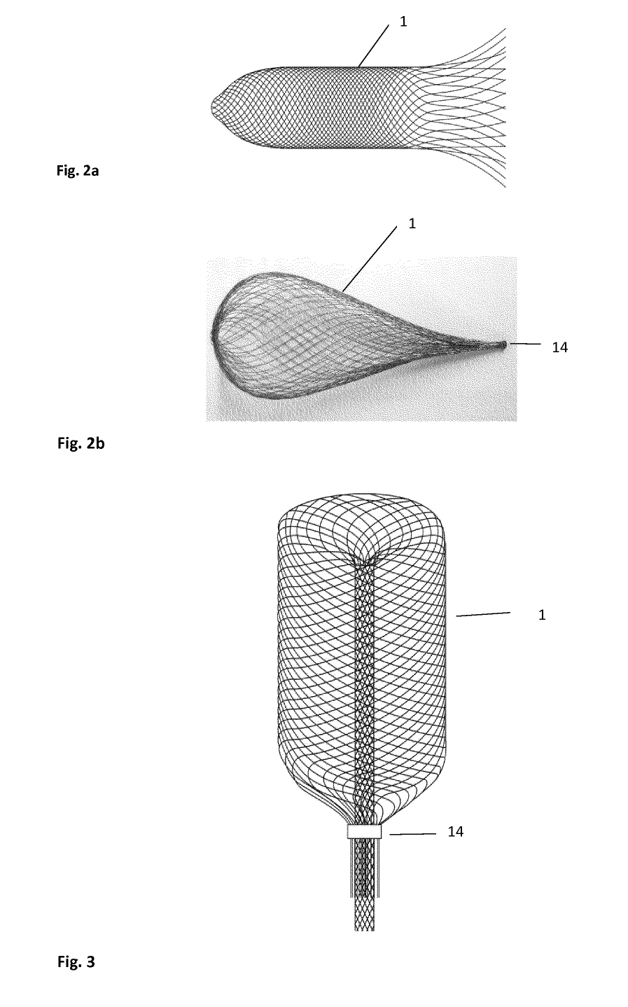

FIGS. 2 and 3 show the braided closure member during its manufacturing before forming the braid.

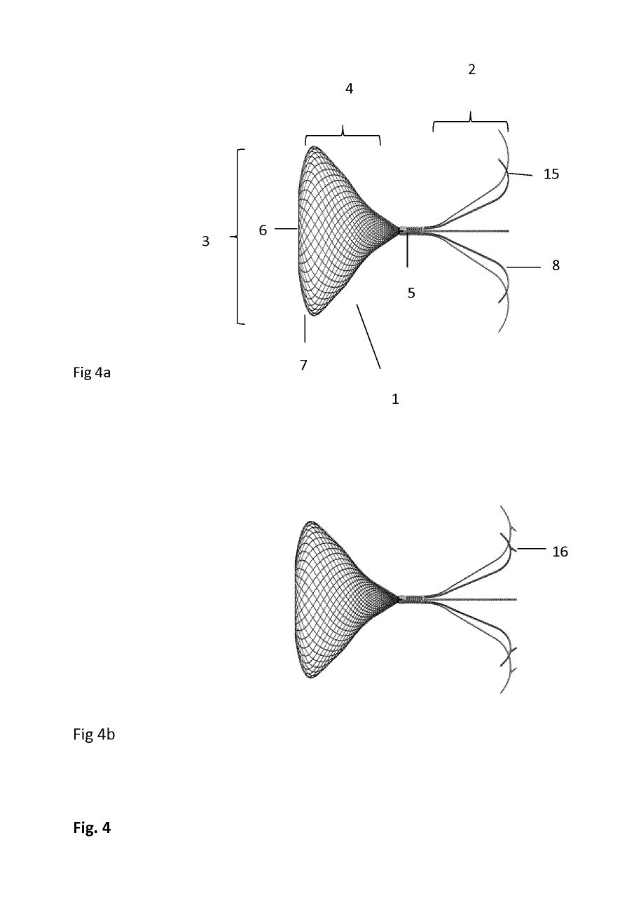

FIG. 4 schematically shows the layout of the closure device.

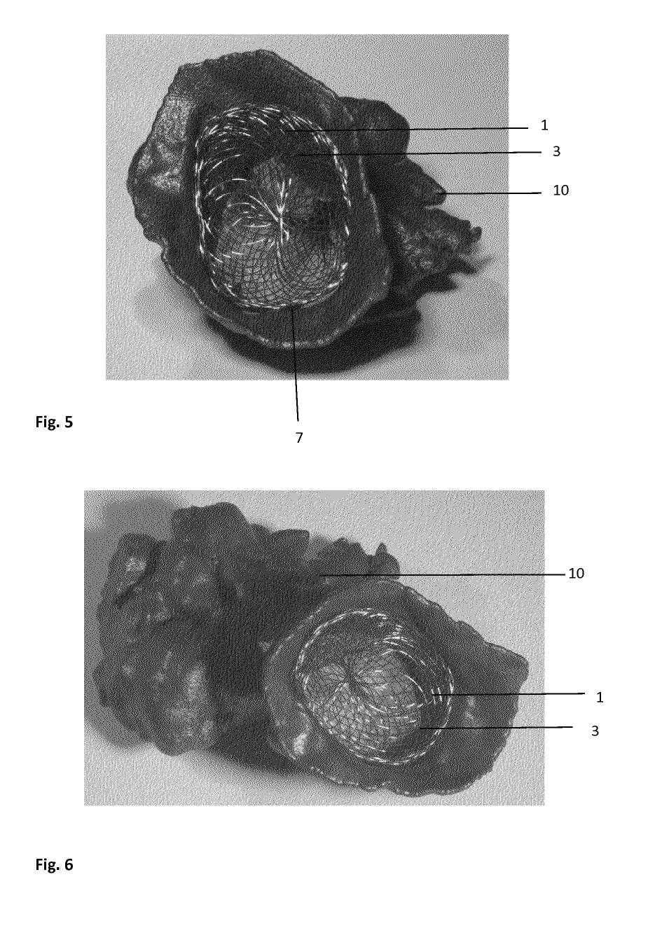

FIGS. 5 to 9 show the forming of the closure member using the LAA model.

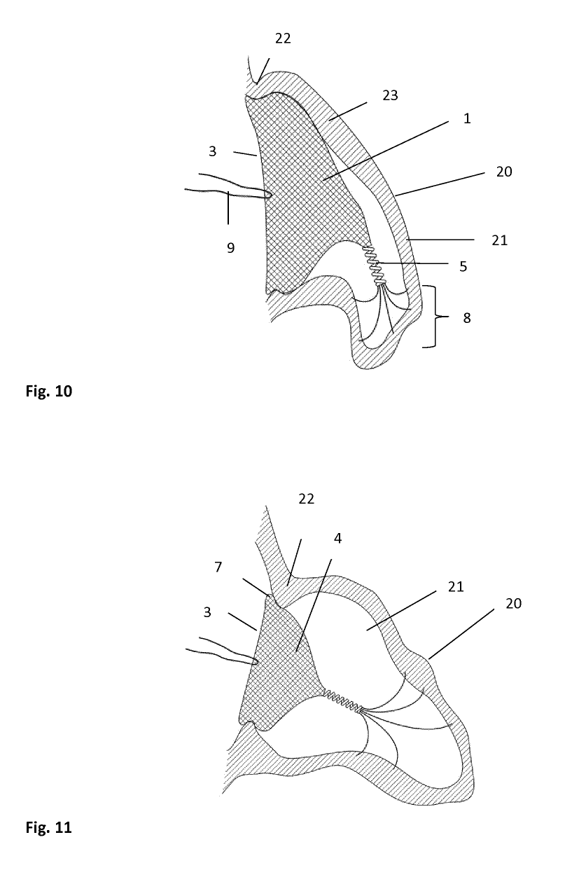

FIGS. 10 to 15 schematically show various embodiments of the braided closure device inserted in the patient's LAA.

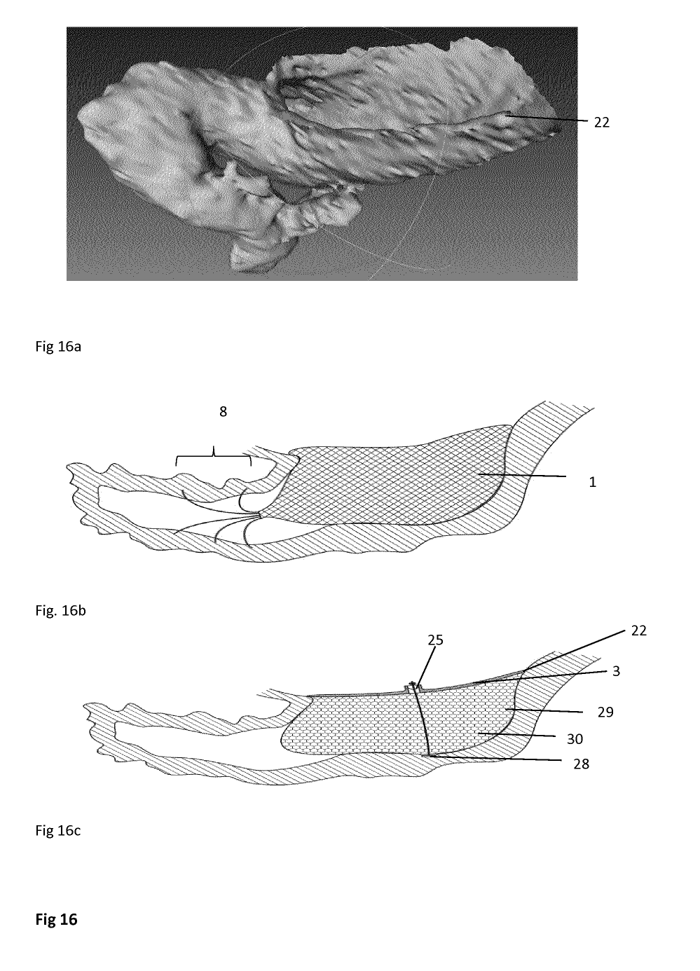

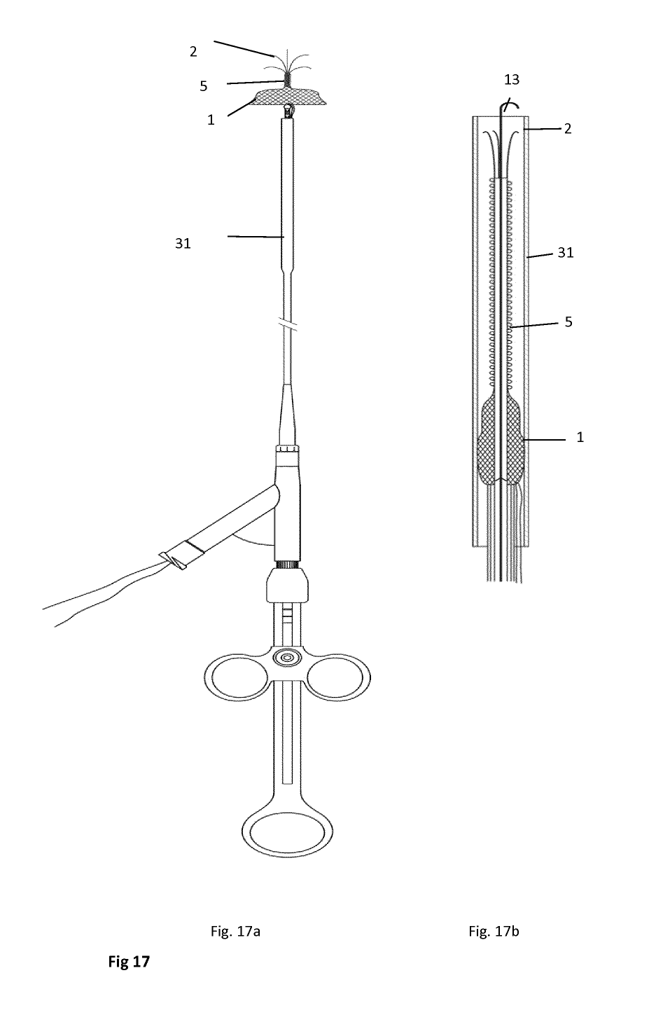

FIG. 16 illustrates the closure of an LAA of the so called chicken wing type using a braid or a polymer device.

FIG. 17 shows the insertion of the closure device via a delivery catheter.

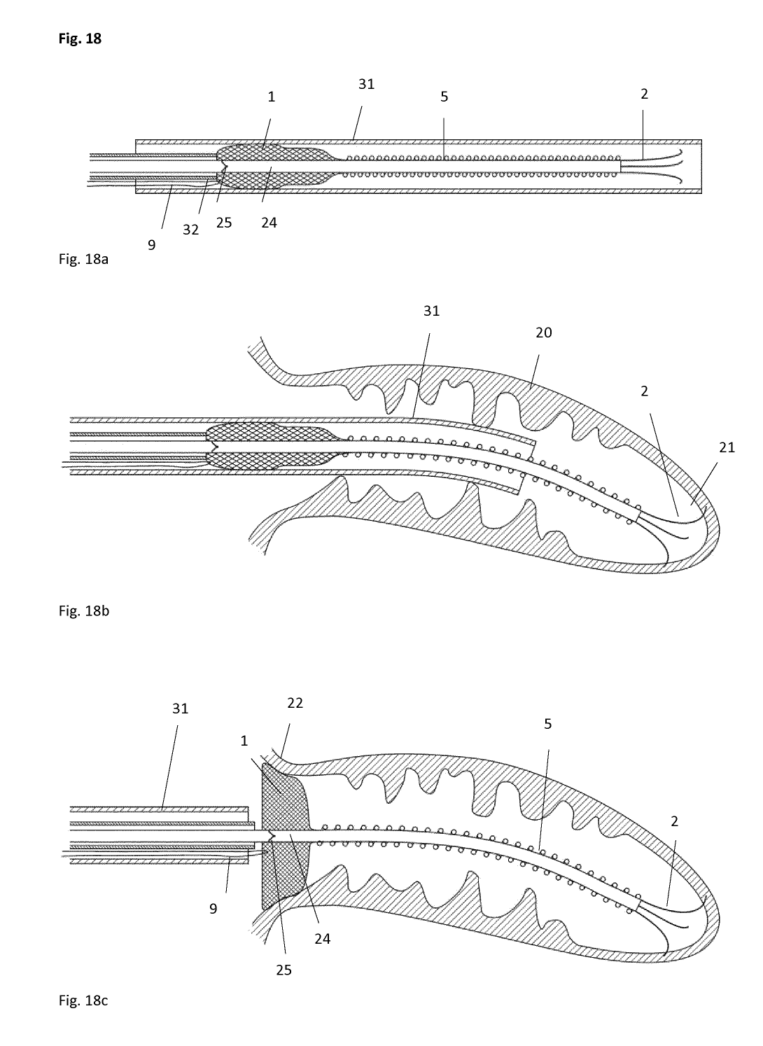

FIG. 18 shows the insertion and implantation process of the closure device.

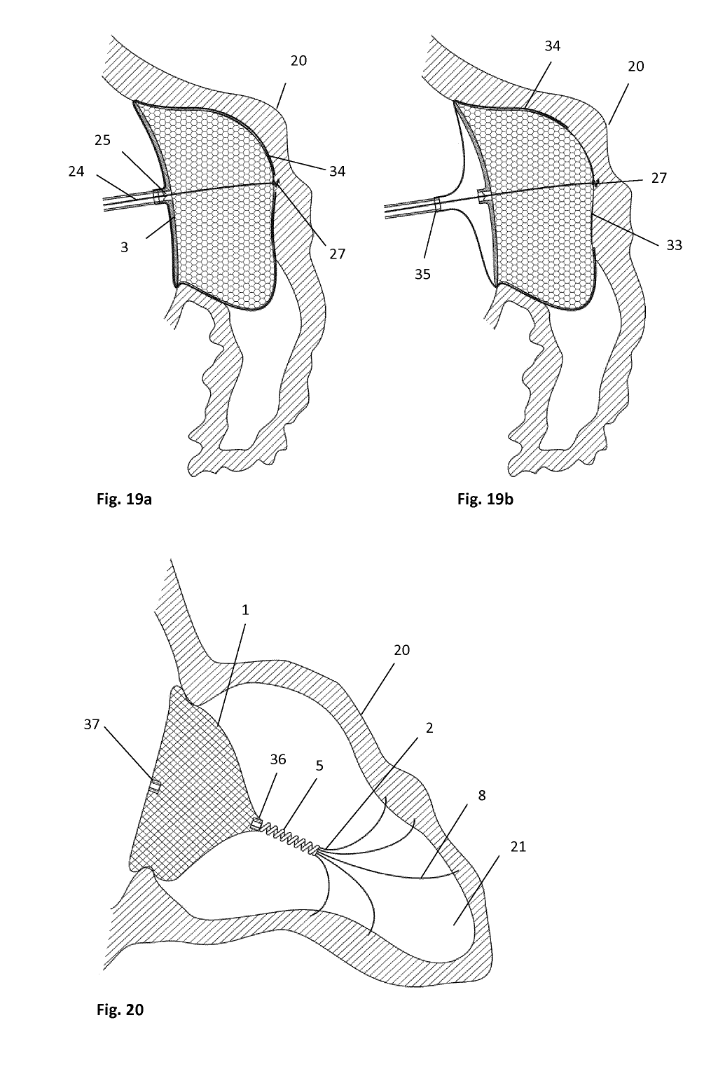

FIG. 19 schematically shows another embodiment of the braided closure device inserted in the patient's LAA.

FIG. 20 schematically shows still another embodiment of the braided closure device inserted in the patient's LAA.

### DETAILED DESCRIPTION OF THE PREFERRED EMBODIMENTS

FIG. 1 is an overview drawing showing a patient's heart with the left atrial appendage (LAA) 100 and the transseptal access to the LAA via the septum 101 and the left atrium 102. A delivery catheter 31 is guided to the LAA. Positioning threads 9 ensure safe positioning of the closure device (not shown).

FIG. 2 shows a braided closure member 1 during its manufacturing. The manufacture of the closure member 1 starts by braiding a core. As shown in FIG. 2a, the manufacture of the closure member 1 may start by braiding a tube-shaped or sleeve-shaped mesh. The ends of the wires of the braided core will then be brought together and connected, for example by a connection part 14, which may be a sleeve or the like. Connection of the wire ends may be achieved without a connection part by directly connecting the wire ends, e.g. by welding, adhesive or other suitable method. As shown in FIG. 2b, the wire ends are connected at the distal end of the closure member 1.

FIG. 3 shows another braided closure member 1 during its manufacturing. In contrast to FIG. 2, the wires of the upper part of the braid are drawn inside the braid, brought together and connected, thus forming a closure member 1 with a double braid structure.

FIG. 4 shows schematically the layout of the closure device before the forming process. The closure device comprises the braided closure member 1 having a lid part 3 and a body part 4, a tension member 5 and a fixing member 2. The lid part 3 has a cover portion 6 and a ring portion 7. The tension member 5 is placed between the closure member 1 and the fixing member 2. The tension member is a coil spring which is fixed to the closure member 1 at its distal end. In this embodiment, the fixing member comprises five wire elements 8 being grouped around the proximal end of the tension member 5. The wire elements 8 are hook-shaped at their distal end. FIG. 4 (i.e. FIGS. 4a, 4b) shows two examples of a hook-shaped layout. FIG. 4a shows a semicircular hook 15. FIG. 4b shows a barbed hook 16.

FIGS. 5 to 9 illustrate the forming of the closure member 1 using a LAA model.

FIG. 5 is a photograph of the LAA model 10 with the braided closure member 1 pressed therein. FIG. 5 mainly illustrates how the lid part 3 of the braided closure member 1 with its ring portion 7 is formed by pressing the braid into the LAA model 10, followed by a heat treatment. It can be seen that the lid part 3 of the closure member 1 conforms in size and shape to the opening of the LAA model 10 and thus to the patient's individual LAA ostium.

FIG. 6 is a photograph of the LAA model 10 in a side view with the braided closure member 1 having a lid part 3 pressed therein. FIG. 6 shows how the complex morphology of the patient's LAA is reproduced by the LAA model 10. Due to the complex anatomy, the length of the fixing member (not shown) has to be adjusted accordingly.

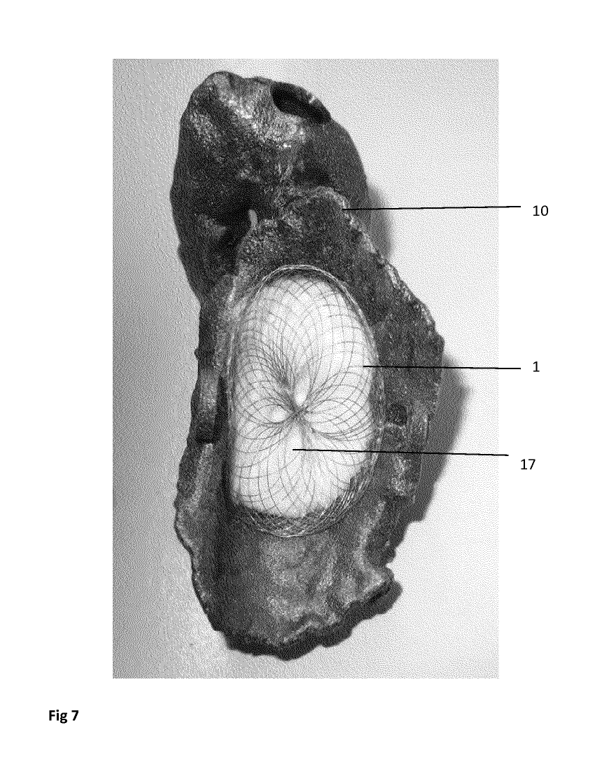

FIG. 7 is a photograph of the LAA model 10 with the braided closure member 1 pressed therein. The braided closure member 1 is filled with heat-resistant filling material such as ceramic wool 17. Due to the ceramic wool, the body part of the braided closure member 1 is pressed against the inner wall of the LAA model 10 to form the body part according to the LAA cavity.

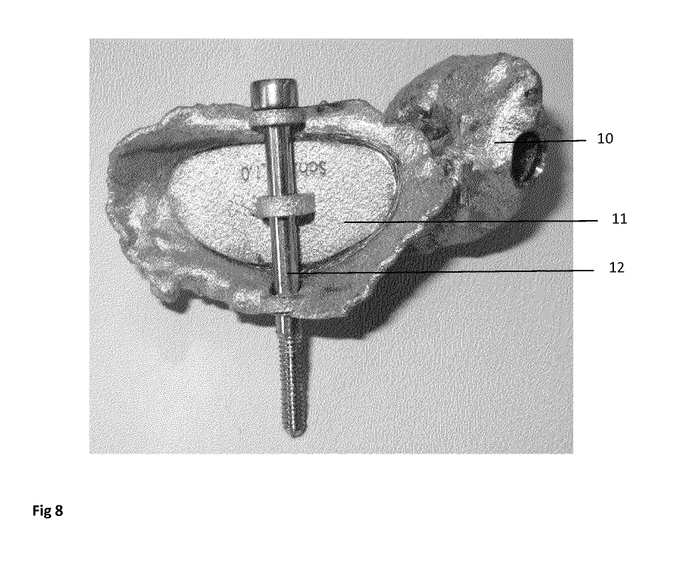

FIG. 8 is a photograph of the LAA model 10 illustrating the fixing of the braided closure member 1 in the LAA model before heat treating. The LAA model 10 is equipped with a metal lid 11 and at least one metal rod 12 to fix the lid 11. It will be appreciated that other mechanisms for fixing the lid 11 are possible. The closed LAA model 10 together with the closure member 1 pressed therein is then heated through the transformation temperatures of the braiding material to attain the shape memory effect. In the case of Nitinol the heating temperature is 400 to 600° C.

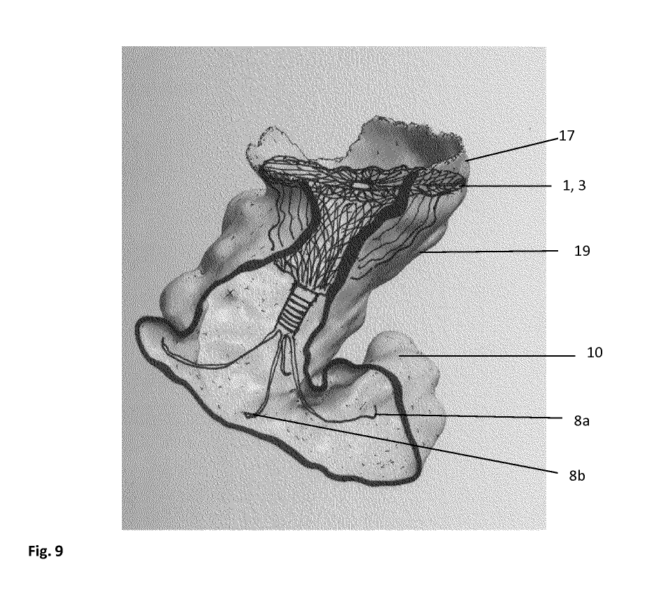

FIG. 9 is a longitudinal three dimensional view of the LAA model 10 with the closure device as shown in FIG. 4 pressed therein after the heat-treatment. The closure device has its final shape. The LAA model is cut open to show how the closure device is arranged therein. The closure member 1 is with its lid part 3 in direct and close contact to the rim 18 of the LAA model 10 and in direct contact to the upper part 19 of the inner wall of the LAA cavity. The wire elements 8a, 8b of the fixing member have different lengths according to the different branches of the LAA cavity. Wire element 8a anchored in an LAA branch is longer than wire element 8b anchored in the central part of the LAA.

FIGS. 10 to 15 show various embodiments of the braided closure device inserted in the patient's LAA. FIG. 10 is a cross section of the patient's LAA 20 with the closure device of FIG. 4 inserted therein. A tension member 5 in the form of a coil spring is attached to the distal end of the braided closure member 1. Wire elements 8 of different length are attached to the coil spring, the wire elements being firmly anchored in the inner wall of the LAA cavity 21. A double filament 9 is attached to the lid part 3 of the closure member 1. The closure member 1 is in direct and close contact to the patient's LAA ostium 22 and to the upper part 23 of the inner wall of the patient's LAA cavity 21. In the case of repositioning, a double filament 9 is attached to the lid part 3 of the closure member 1 to withdraw the closure member 1 back into the delivery catheter. The filament 9 continues to the proximal end of the handle of the delivery catheter (see FIG. 1). After the final position of the closure device is reached, the double filament 9 is completely withdrawn from the closure device by pulling at one filament.

FIG. 11 is a cross section of the patient's LAA 20 with the closure device of FIG. 4 inserted therein. In contrast to FIG. 10, there is shown a braided closure member 1 whose body part 4 is not in direct contact to the tissue of the patient's LAA cavity 21. The lid part 3 of the closure member 1 with ring part 7 seals the LAA ostium 22.

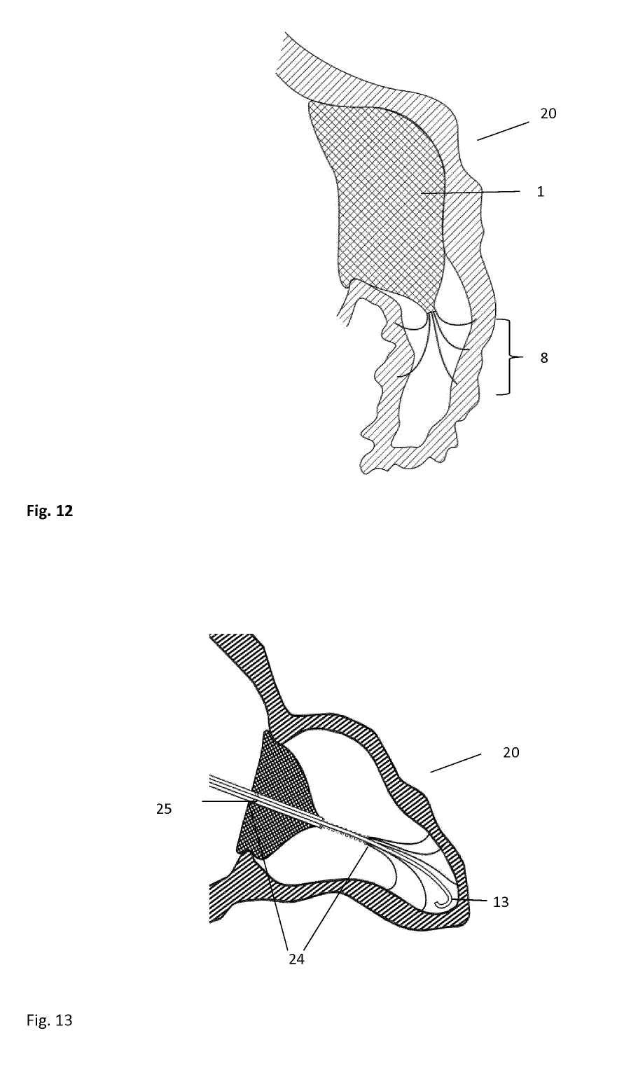

FIG. 12 is a cross section of the patient's LAA 20 with a further embodiment of the closure device inserted therein. In contrast to FIG. 10, this alternative closure device has no tension member between closure member 1 and fixing member 2, the wire elements 8 of the fixing member 2 thus being directly grouped around the distal end of the braided closure member 1.

FIG. 13 is a cross section of the patient's LAA 20 with the closure device of FIG. 4 inserted therein as illustrated in FIG. 11. In addition to the device shown in FIG. 11, a guide wire 13 has been placed before insertion of the closure device to facilitate insertion. The closure member 1 and the tension member 5 have a central lumen 24 extending through the device and equipped with a valve 25 to seal the lumen 24. The lumen 24 provides the possibility of using a guidewire or guiding catheter.

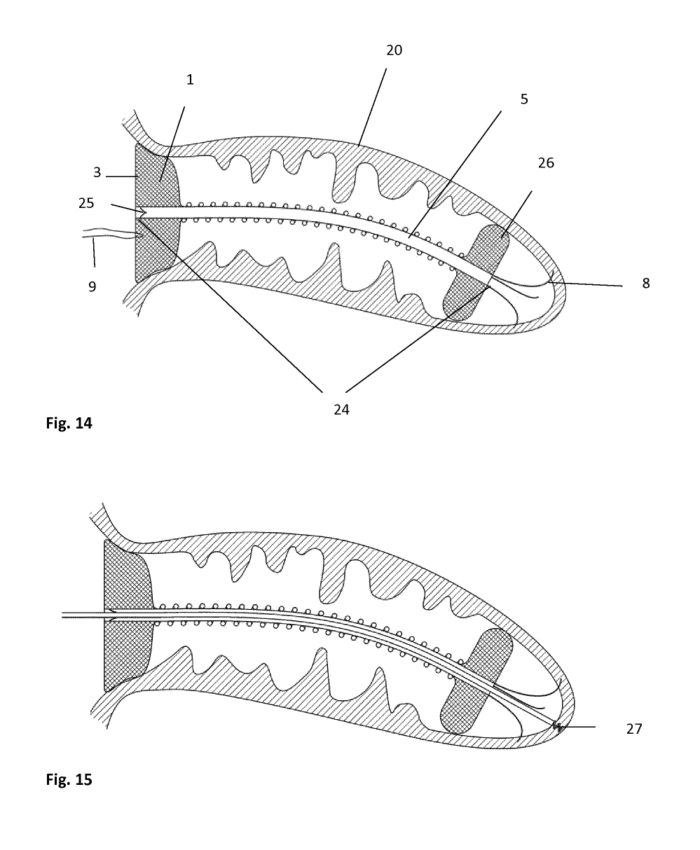

FIG. 14 is a cross section of the patient's LAA 20 with a further embodiment of the closure device inserted therein. The closure device comprises a first closure member 1, a tension member 5 being distally attached to the closure member 1, and a double filament 9 being attached to the lid part 3 of the closure member 1. Between the tension member 5 and the fixing member 2 there is an additional braid functioning as an additional closure disc 26. The wire elements of the fixing member 2 are grouped around the distal end of the additional closure disc 26. The closure member 1 and the tension member 5 have a central lumen 24 extending through the device and equipped with a valve 25 to seal the lumen 24. The lumen 24 provides the possibility of placing a guide wire or guiding catheter as shown in FIG. 13 or of inserting anchoring members as shown in FIG. 15.

FIG. 15 is a cross section of the patient's LAA 20 with the closure device of FIG. 14 inserted therein. In contrast to the device shown in FIG. 14, a screw-in type coil 27 acting as an additional fixing member has been inserted.

FIG. 16 illustrates the closure of an LAA of the so called chicken wing type. The LAA ostium 22 is elongated and narrow and the LAA lobe is of the chicken wing type with an obvious bend in the end part. FIG. 16 makes the advantage of the invention particularly apparent, as the bent lobe with the elongated opening cannot be sealed with known occluders which are mainly circular.

FIG. 16a is a computer graphic made on the basis of the patient's LAA. FIG. 16b is the corresponding cross section with a Nitinol braid, as described in FIG. 12, inserted therein.

Regarding FIG. 16c, the closure member 1 is formed of biocompatible plastic. The fixing member is a T-bar 28 which is positioned at the distal end of the closure member 1. The closure member 1 comprises a lid part 3 and a body part in the form of a balloon 29, said balloon 29 being in close and direct contact to the patient's LAA cavity. The balloon 29 is filled with liquid or foam 30 made of silicone or polyurethane. The lid part 3 comprises a cover portion and a ring portion formed according to the patient's LAA ostium and sealing the LAA ostium 22. The lid part 3 and the balloon 29 have a central lumen extending through the device and equipped with a valve 25 to seal the lumen.

FIG. 17 shows how the closure device is guided into the heart. FIG. 17a shows the delivery catheter 31 with the closure member 1, fixing member 2 and tension member 5 in the release position. FIG. 17b is a cross section of the delivery catheter 31 showing the folded closure member 1, the tension member 5 and the fixing member 2. Guide wire 13 facilitates the placement of the device.

With continuing reference to FIG. 18, an exemplary insertion method is described. It will be appreciated that the method can be carried out using a guide wire as described in connection with FIG. 17. FIG. 18a shows a cross-section of the delivery catheter with the closure device in an initial configuration. In particular, the closure member 1 is in a compressed configuration. Likewise, the fixation member 2 is in a compressed configuration. The tension member connects the closure member 1 and the fixation member 2. The insertion catheter 31 is open at its distal end so that the closure device can be advanced from the catheter 31 by means of a pusher 32. The valve 25 prevents blood from flowing in a proximal direction through the lumen of the closure device. The closure device is completely pushed out of the insertion catheter 31 by means of the pusher 32. A thread 9 can be used to reposition the closure device if necessary.

FIG. 18b shows the insertion catheter 31 inserted into the patient's LAA 20. The fixation elements 2 have been pushed out of the catheter 31 and radially expanded. Because of their hook-shaped ends they have anchored to the internal wall of the LAA cavity 21. The closure device is secured to the LAA 20 and the tension member 5 is about to exit the catheter 31.

FIG. 18c shows the progress of implantation. The insertion catheter 31 has been further retracted and the braided closure member 1 has expanded. The lumen 24 is closed by the valve 25. Because the fixation elements 2 are anchored to the internal wall of the patient's LAA 20, the tension member 5 is tensioned by pulling the closure member 1 in a proximal direction during retraction of the catheter 31. This tension causes the closure member 1 to be biased against the LAA ostium 22 to thereby close and seal the LAA ostium 22. If desired or necessary, the closure member 1 can be repositioned with the help of the thread 9 by pulling the closure member 1 out of the ostium 22, rotating the closure member 1 and releasing the thread 9. The thread 9 may be a double thread and is removed after implantation of the closure device.

FIG. 19a is a cross section of the patient's LAA 20 with another embodiment of a closure device inserted therein. The fixing member is an adhesive layer 33 of a tissue adhesive being applied to the surface of the closure device. A protective layer 34 which is applied to the surface of the adhesive layer 33 protects the closure device from being glued to the LAA tissue during the insertion of the device. The lid 3 has a central lumen 24 extending through the device equipped with a valve 25 to seal the lumen 24. A screw-in type coil 27 acting as temporary fixing member is inserted through said lumen 24.

FIG. 19b shows the removal of the protective layer 34 and the gluing process. The protective layer 34 which is attached to a ring 35 is removed by pulling the ring 35 to thereby successively release the adhesive layer 33 to create an adhesive joint between the surface of closure device and the LAA tissue. After the adhesive process has been completed the temporary fixing member (coil spring 27) is removed.

FIG. 20 is a cross section of the patient's LAA 20 with another embodiment of a closure device inserted therein. A tension member 5 in the form of a coil spring is attached to the distal end of the braided closure member 1. Wire elements 8 with different lengths are attached to the coil spring 5, the wire elements 8 being firmly anchored into the inner wall of the LAA cavity 21. The lid has a connector 37 for a cable, such as an internal thread, so that an extension cable (not shown) can be attached to the closure device acting as an indifferent pole for a pacemaker or cardioverter. At the distal end of the closure device, between the closure member 1 and the coil spring 5, there is a bearing 36 so that the closure member 1 can be rotated in order to allow fine adjustment of the closure member 1 without removing the wire elements 8. It will be appreciated that the connector for a cable or the bearing may be included also in the other embodiments.

Some preferred embodiments of the invention are described in the following enumerated items 1 to 12. However, the invention is defined in the appended claims.
1. A medical closure device that is suitable for closing the left atrial appendage (LAA) consisting of a braid made of a shape memory material, a tension spring and at least one fixation element. The braid is proximally disc-shaped such that a closure disc is provided proximally, which conforms in shape to the anatomy of the ostium in the target LAA true to scale. The braid is distally connected to a tension spring, which has distally at least one fixation element.
2. The closure device according to item 1 formed of Nitinol.
3. The closure device according to item 1 or 2, wherein the braid is completely or partially covered with a plastic membrane on the outside or the inside.
4. The closure device of item 1, wherein the tension spring is made of Nitinol.
5. The closure device of item 1, wherein the fixation element is made of a Nitinol wire and formed hook-shaped at its distal end.
6. The closure device of item 1 or 5, wherein 3-10 fixation elements are provided which are grouped radially about the virtually extended central axis of the tension spring.
7. The closure device of item 6, wherein 4 Nitinol wires are provided having a distance of about 90 degrees from each other.
8. The closure device according to any one of items 1 to 7 having a central lumen and a self-sealing valve sealing the lumen to the outside.
9. A method of manufacturing a closure device that is configured to be inserted into a patient's LAA (left atrial appendage) cavity, the method comprising the following steps:
a) providing a computer generated three-dimensional model of the LAA based on medical images of the LAA,
b) transferring the data of the computer generated model to a 3-D printer device,
c) manufacturing a real model by selective laser sintering,
d) manufacturing a braid of a shape memory material and shaping the braid by pressing the braid into the model obtained in step c) or a subsequent model, wherein a braid is created by the pressing, which is formed proximally disc-shaped, such that a closure disc is created, which conforms in shape to the anatomy of the ostium in the target LAA true to scale;
e) tempering the braid for fixation of the shape change;
f) capturing the ends of the braid in a sleeve and connecting the braid with the tension spring, which has distally at least one fixation element.
10. A closure device manufactured according to a method described in item 9.
11. A braid of a shape memory material that is formed proximally disc-shaped so that a closure disc is provided, which conforms in shape to the anatomy of the ostium in the target atrial appendage true to scale.
12. An insertion catheter consisting of an insertion catheter, into which the closure device of the items 1 to 8 is inserted in a compressed configuration.

Further preferred embodiments are described in the enumerated following items 13 to 36, wherein the closure device as defined in the following items does not comprise at least one, any, various or all of the features of the closure device as defined in any one of the above items 1 to 8, 10 and 11. In a particularly preferred embodiment, the closure device defined in the following items does not comprise a tension spring. Likewise, the method as defined in the following items does not comprise at least one, any, various or all of the features of the method as defined in the aforementioned item 9. In a particularly preferred embodiment, in the method defined in the following items the LAA model is not manufactured by selective laser sintering.
13. A medical closure device that configured to be inserted into a patient's LAA (left atrial appendage) cavity, comprising a closure member and at least one fixing member. The closure member comprises a lid part and a body part one following the other in a proximal-distal direction. The lid part comprises a cover portion and a ring portion, the ring portion being configured to be circumferentially disposed along the rim of an individual LAA ostium, thus sealing the LAA ostium completely.
14. The closure device according to item 13, wherein the closure device is formed of metal or biocompatible polymer or a combination thereof.
15. The closure device according to item 13 or 14, wherein the closure device is formed of shape memory material.
16. The closure device according to any one of items 13 to 15, wherein the closure device is formed of Nitinol.
17. The closure device according to any one of items 13 to 16, wherein the body part of the closure member is configured to adjoin the tissue of an individual LAA cavity.
18. The closure device according to any one of items 13 to 17, wherein the closure member is a metal braid.
19. The closure device according to any one of items 13 to 18, wherein the closure member comprises a body part in the form of a polymer balloon and a lid part made of a polymer or of a metal braid.
20. The closure device according to item 18 or 19, wherein the braid comprises at least one layer of occluding material in the form of a coating or a patch.
21. The closure device according to any one of items 13 to 20, wherein the fixing member is hook-shaped and comprises at least one wire element or tape element made of shape memory material.
22. The closure device according to any one of items 13 to 21, wherein the fixing member comprises 2 to 10 wire elements, preferably 3 to 6 wire elements, connected to and grouped evenly around the distal end of the body part of the closure member.
23. The closure device according to item 22, wherein the wire elements of the fixing member have different lengths with respect to one another.
24. The closure device according to any one of items 13 to 23, further comprising a tension member placed between the closure member and the fixing member.
25. The closure device according to item 24, wherein the fixing member comprises two to ten wire elements, preferably three to six wire elements, connected to and grouped evenly around the distal end of the tension member.
26. The closure device according to item 24 or 25, wherein the tension member is a coil spring.
27. The closure device according to any one of items 13 to 26, further comprising a thread or double thread at the proximal end of the closure member.
28. The closure device according to any one of items 1 to 27, wherein the closure member has a central lumen extending through the lid part and the body part with a valve placed therein, said valve sealing the lumen.
29. The closure device according to item 13, wherein the whole closure device is made of Nitinol, including the closure member, the fixing member and a tension member, wherein the closure member is a braid comprising at least 30 wire elements having a diameter of about 0.03 to 0.15 mm, said braid defining the lid part and the body part; wherein the fixing member comprises two to ten wire elements grouped evenly around a distal end of the tension member; wherein the tension member is a coil spring, said coil spring being attached to the distal end of the body part of the closure member.
30. A method of manufacturing a closure device that is configured to be inserted into a patient's LAA (left atrial appendage) cavity, preferably the closure device of any one of items 13 to 29, the method comprising the following steps:
a) generating a three-dimensional computer graphic of the patient's left atrial appendage (LAA) based on medical images of the LAA,
b) transferring the data of the computer graphic to a 3-D printer device,
c) forming an LAA model by 3-D printing, said model being an exact reproduction of the patient's LAA,
d) manufacturing the closure device by pre-fabricating a closure member comprising a lid part and a body part one following the other in a proximal-distal direction and pre-fabricating a fixing member and forming at least the lid part of the closure member according to the LAA model obtained in step c) to obtain a pre-formed closure device with at least the lid part of the closure member conforming in size and shape to the patient's LAA ostium; and attaching the fixing member to the closure member either before forming the closure member or thereafter.
31. A method of manufacturing a closure device that is configured to be inserted into a patient's LAA (left atrial appendage) cavity, preferably the closure device of any one of items 13 to 29, the method comprising the following steps:
a) generating a three-dimensional computer graphic of the patient's left atrial appendage (LAA) based on medical images of the LAA,
b) transferring the data of the computer graphic to a 3-D printer device,
c) forming an LAA model by 3-D printing, said model being an exact reproduction of the patient's LAA,
d) manufacturing the closure device by pre-fabricating a closure member comprising a lid part and a body part one following the other in a proximal-distal direction, and pre-fabricating a fixing member, filling the closure member with a heat-resistant material and forming the closure member by placing it into the LAA model obtained in step c) to obtain a pre-formed closure device which conforms in size and shape to the patient's LAA ostium and the LAA cavity; and attaching the fixing member to the closure member either before forming the closure member or thereafter.
32. A method of manufacturing a closure device that is configured to be inserted into a patient's LAA (left atrial appendage) cavity, preferably the closure device of any one of items 13 to 29, the method comprising the following steps:
a) generating a three-dimensional computer graphic of the patient's left atrial appendage (LAA) based on medical images of the LAA,
b) transferring the data of the computer graphic to a 3-D printer device,
c) forming an LAA model by 3-D printing, said model being an exact reproduction of the patient's LAA,
d) manufacturing the closure member and forming the closure member by first forming a metal ring according to the opening of the LAA model and fixing the closure member to the metal ring to shape it.
33. The method according to any one of items 30 to 32, wherein the medical image is an X-ray image, a CT scan, an MRI scan, or an ultrasound image.
34. The method according to any one of items 30 to 33, wherein 3-D printing is 3-D laser printing and the LAA model is made of metal.
35. The method according to any one of items 30 to 33, wherein 3-D printing is 3-D polymer printing and the LAA model is made of polymer.
36. The method according to any one of items 30 to 35, wherein the closure device is made of a shape memory alloy, the method further comprising the following step e):
e) heat-treating the LAA model together with the closure member or closure device placed therein to the transformation temperature of the shape memory alloy to obtain a pre-formed closure device with at least the lid part of the closure member conforming in size and shape to the patient's LAA ostium.

1. A medical closure device configured to be inserted into a patient's left atrial appendage (LAA) cavity, comprising a closure member and at least one fixing member, the closure member comprising a lid part and a body part one following another in a proximal-distal direction, the lid part comprising a cover portion and a ring portion, the ring portion being configured to be circumferentially disposed along a rim of an individual LAA ostium, thus sealing the LAA ostium completely.

2. The closure device according to claim 1, wherein the closure device is formed of metal or biocompatible polymer or a combination thereof.

3. The closure device according to claim 1, wherein the closure device is formed of shape memory material.

4. The closure device according to claim 1, wherein the closure device is formed of Nitinol.

5. The closure device according to claim 1, wherein the body part of the closure member is configured to adjoin tissue of an individual LAA cavity.

6. The closure device according to claim 1, to wherein the closure member is a metal braid.

7. The closure device according to claim 1, wherein the closure member comprises a body part in a form of a polymer balloon and a lid part made of a polymer or of a metal braid.

8. The closure device according to claim 6, wherein the braid comprises at least one layer of occluding material in a form of a coating or a patch.

9. The closure device according to claim 1, wherein the fixing member is hook-shaped and comprises at least one wire element or tape element made of shape memory material.

10. The closure device according to claim 1, wherein the fixing member comprises 2 to 10 wire elements, preferably 3 to 6 wire elements, connected to and grouped evenly around [[the]] a distal end of the body part of the closure member.

11. The closure device according to claim 10, wherein the wire elements of the fixing member have different lengths with respect to one another.

12. The closure device according to claim 1, further comprising a tension member placed between the closure member and the fixing member.

13. The closure device according to claim 12, wherein the fixing member comprises 2 to 10 wire elements, preferably 3 to 6 wire elements (8), connected to and grouped evenly around a distal end of the tension member.

14. The closure device according to claim 12, wherein the tension member is a coil spring.

15. The closure device according to claim 1, further comprising a thread or double thread at a proximal end of the closure member.

16. The closure device according to claim 1, wherein the closure member has a central lumen extending through the lid part and the body part with a valve placed therein, said valve sealing the lumen.

17. The closure device according to claim 1, wherein the whole closure device is made of Nitinol, including the closure member, the fixing member and a tension member, wherein the closure member is a braid comprising at least 30 wire elements having a diameter of about 0.03 to 0.15 mm, said braid defining the lid part and the body part, wherein the fixing member comprises 2 to 10 wire elements grouped evenly around a distal end of the tension member, wherein the tension member is a coil spring, said coil spring being attached to the distal end of the body part of the closure member.

18. A method of manufacturing a closure device that is configured to be inserted into a patient's left atrial appendage (LAA) cavity, the method comprising the following steps:
a) generating a three-dimensional computer graphic of the patient's (LAA based on medical images of the LAA;
b) transferring data of the computer graphic to a 3-D printer device;
c) forming an LAA model by 3-D printing, said model being an exact reproduction of the patient's LAA; and
d) manufacturing the closure device by pre-fabricating a closure member comprising a lid part and a body part one following another in a proximal-distal direction and pre-fabricating a fixing member and forming at least the lid part of the closure member according to the LAA model obtained in step c) to obtain a pre-formed closure device with at least the lid part of the closure member conforming in size and shape to the patient's LAA ostium, and attaching the fixing member to the closure member either before forming the closure member or after forming the closure member.

19. A method of manufacturing a closure device that is configured to be inserted into a patient's left atrial appendage (LAA) cavity, the method comprising the following steps:
a) generating a three-dimensional computer graphic of the patient's (LAA based on medical images of the LAA;
b) transferring the data of the computer graphic to a 3-D printer device;
c) forming an LAA model by 3-D printing, said model being an exact reproduction of the patient's LAA; and
d) manufacturing the closure device by pre-fabricating a closure member comprising a lid part and a body part one following another in a proximal-distal direction, and pre-fabricating a fixing member, filling the closure member with a heat-resistant material and forming the closure member by placing it into the LAA model obtained in step c) to obtain a pre-formed closure device which conforms in size and shape to the patient's LAA ostium and the LAA cavity, and attaching the fixing member to the closure member either before forming the closure member or after forming the closure member.

20. A method of manufacturing a closure device that is configured to be inserted into a patient's left atrial appendage (LAA) cavity, the method comprising the following steps:
a) generating a three-dimensional computer graphic of the patient's (LAA based on medical images of the LAA;
b) transferring the data of the computer graphic to a 3-D printer device;
c) forming an LAA model by 3-D printing, said model being an exact reproduction of the patient's LAA; and
d) manufacturing the closure member and forming the closure member by first forming a metal ring according to an opening of the LAA model and fixing the closure member to the metal ring to shape it.

21. The method according to claim 18, wherein the medical image is an X-ray image, a CT scan, an MRI scan, or an ultrasound image.

22. The method according to claim 18, wherein 3-D printing is 3-D laser printing and the LAA model is made of metal.

23. The method according to claim 18, wherein 3-D printing is 3-D polymer printing and the LAA model is made of polymer.

24. The method according to claim 18, wherein the closure device is made of a shape memory alloy, the method further comprising step e) comprising:
e) heat-treating the LAA model together with the closure member or closure device placed therein to a transformation temperature of the shape memory alloy to obtain a pre-formed closure device with at least the lid part of the closure member conforming in size and shape to the patient's LAA ostium.
